(19) United States
(12) Patent Application Publication
(10) Pub. No.: US 2009/0060894 A1
(43) Pub. Date: Mar. 5, 2009

(54) Treatment to aid in the metabolism of alcohol

(76) Inventors: John Charin Somberg, Lake Forest, IL (US); Vasant V. Ranade, Libertyville, IL (US)

Correspondence Address: John C. Somberg, MD, 210 So. Saunders Road, Lake Forest, IL, 60045, US

(21) Appl. No.: 11/895,655

(22) Filed: Aug. 27, 2007

(51) Int. Cl. A61K 38/44; A61P 3/00

(52) U.S. Cl. 424/94.4

(57) ABSTRACT

Alcohol intoxication is a serious problem. Many individuals are more sensitive to alcohol intoxication and placed at a decided disadvantage in social drinking situations. What is proposed is a way to facilitate alcohol metabolism in individuals who have consumed alcohol by the administration of enzymes to speed the alcohols metabolism.

### REFERENCES CITED

### U.S. Patent Documents

| | | | |
|---|---|---|---|
| 5,783,189 | July 1998 | Pie et al | 424/195.1 |
| 6,120,805 | September 2000 | Whitmire | 424/497 |
| 6,255,497 | July 2001 | Vallee et al | 549/403 |
| 6,465,436 | October 2002 | Lukas et al | 514/27 |
| 4,165,376 | August 1979 | Rosenberg | 424/267 |
| 5,053,396 | October 1991 | Blass | 514/45 |
| 4,450,153 | May 1984 | Hopkins | 424/94 |
| 5,324,516 | June 1994 | Pek et al | 424/195.1 |
| 5,547,671 | August 1996 | Duthinh | 424/195.1 |
| 5,888,532 | March 1999 | Pritsos et al | 424/423 |
| 5,559,152 | September 1996 | Komissarova et al | 514/557 |

### BACKGROUND OF THE INVENTION

Alcohol intoxication is a serious problem. Individuals may exceed their alcohol intake capacity and become inebriated by design or accident. Accidental alcohol intoxication may lead to embarrassing social circumstances and even ruinous events. The individual who exceeds the legal alcohol limit, even without signs of impairment, may become involved in an auto accident, tested, found intoxicated and criminally prosecuted. The unpredictable nature of potential alcohol intoxication is a major public health problem. This is especially the case in individuals who are more susceptible to the intoxicating effects of alcohol. Individuals may not experience the enjoyable effects of alcohol, but rather be subject to nausea, vomiting and signs of Central Nervous System (CNS) intoxication. Besides individual variability in alcohol adverse response, there is a known increased sensitivity by certain groups. Many individuals of Asian decent show an increased susceptibility to alcohol toxicity. In addition, there is an extensive literature to the effect that women are more susceptible to alcohol toxicity than men.

The increased sensitivity of women to alcohol toxicity may result form a reduced enzymatic capacity in the Gastro-Intestinal (GI) system to initially metabolize alcohol. This genetic susceptibility places women at times in difficult social circumstances, increasing the potential for alcohol intoxication. The enhanced potential for alcohol intoxication can be likened to individuals with a lactose deficiency who develop severe abdominal pain, nausea, vomiting and/or diarrhea when they ingest milk products.

Alcohol is metabolized both in the stomach, small intestine and in the liver by alcohol dehydrogenase and the product of this enzymatic reaction, an aldehyde is then metabolized by aldehyde dehydrogenase. The second step is most important, since the aldehyde is a toxic intermediate and if it were to build up in the body this would lead to adverse consequences, especially CNS toxicity.

To moderate the adverse build up of alcohol or aldehyde in the body following alcoholic beverage ingestion, it is suggested that a mixture of alcohol dehydrogenase and aldehyde dehydrogenase enzymes are combined to be taken prior or with an alcohol beverage to reduce the blood alcohol or aldehyde concentration that can lead to alcohol intoxication.

### PRIOR ART

A number of approaches have been proposed to deal with the problem of alcohol intoxication. None thought have suggested the subject of this patent the exogenases administration of the alcohol metabolizing enzymes with appropriate emoluments to facilitate alcohol metabolism.

One approach has been to facilitate the avoidance of alcohol beverages. Pei and associates have proposed the use of Puevaria labata and derivative thereof for treating alcohol dependence. (U.S. Pat. No. 5,783,189 to Pei, Overstreet, Rezvani and Lee). U.S. Pat. No. 6,120,806 to Whitmore, teaches that an oral controlled release dosage form for cyanamide when administered to ethanol metabolizing individuals can elevate blood acetaldehyde to such levels and for such periods of time, that the individuals will be deterred from future alcohol consumption. Vallee and Keung propose an invention using daidzein or similar ALDH inhibiting compound such as a synthetic analog of daidzein for the inhibition of aldehyde dehydrogenase facilitating the avoidance of alcohol (U.S. Pat. No. 6,255,497 to Vallee and Keung). Another method for the avoidance of alcohol is proposed by Lukas and Lee that involves the administration of a pharmaceutical containing the extract of the kudzu plant, Puevaria lobata (Kudzu plant). (U.S. Pat. No. 6,465,436 B2 to Lukas and Lee). The kudzu extract has been shown to reduce the desire of heavy drinkers to consume alcohol.

Other approaches have focused on the use of antagonists of alcohol effects using antagonists of 5-hydroxy-tryptamine alone (U.S. Pat. No. 4,165,376 to Rosenberg) or combined with other agents (U.S. Pat. No. 5,053,396 to Blass) to facilitate its actions. However, the majority of approaches have focused on the enzymatic pathways involved in alcohol degradation. Alcohol is metabolized first to an aldehyde by alcohol dehydrogenase with NAD. The aldehyde is then converted by aldehyde dehydrogenase using NAD and water in the reaction. The initial conversion is much slower and is the rate limiting step. U.S. Pat. No. 4,450,153 to Hopkins proposes to reduce the level of alcohol content in blood by contacting the blood alcohol with the enzyme alcohol oxidase. The invention concerns the exposure of blood by injection, extra corporeal shunt (dialysis) and oral administration. However, the invention does not deal with the problems of aldehyde buildup and toxicity, rapid gastric emptying or gastric acid pH inactivating the enzyme(s). Others had taught a method to increase the activity of the metabolic enzymes involved in alcohol metabolism. U.S. Pat. No. 5,324,516 to Pek, Kim, Hwang, Park, Kyonggi and Kwon teaches that a galenic coposition comprising an amount of fructose and an aqueous extract of pueraria flower, phaseoli radiati semen and pinelliae tuber sufficient to increase, in vivo, metabolic activity of alcohol dehydrogenase and aldehyde dehydrogenase. U.S. Pat. No. 5,547,671 to Duthinh teaches that vegetable extracts in defined quantities, at lease one of them containing naturally occurring daidzin and daidzein in sufficient quantities to facilitate gastric and hepatic metabolism of alcohol. U.S. Pat. No. 5,888,532 to Pritsos and Miller propose to use pyridine nucleotide phosphate derivatives. U.S. Pat. No. 5,559,152 to Komissarova and associates proposes the use of succinic acid in combination with citric acid to prevent alcohol intoxication.

None of the proposed approaches directly or indirectly proposes to enhance alcohol metabolism by having individuals ingest exogenesis enzymes specifically alcohol and aldehyde dehydrogenase along with anti-cholergenic and antacid compounds to facilitate their action.

### SUMMARY OF THE INVENTION

Specifically, a treatment is suggested consisting of a mixture of alcohol and aldehyde dehydrogenase that can be taken orally to aid in the metabolism of alcohol.

### BRIEF DESCRIPTION OF INVENTION

To establish the feasibility of enzymatic reduction of alcohol concentration enzymatic studies in vitro were undertaken. The hypothesis was that a stock solution of ethyl alcohol at a pre-determined concentration would have the ethyl alcohol concentration reduced when mixed with alcohol dehydrogenase and aldehyde dehydrogenase. In addition, nicotinamide adenine dinudeotide (NAD), sodium prophasphate buffer pH 3.5 were added, since an in vitro solution requires the phosphate and NAD for the enzymatic activity of alcohol dehydrogenase enzyme, cofactors present in vivo.

### EXAMPLE

Group I

ETOH+NAD+Na pyrophosphate+Na phosphate+alcohol dehydrogenase

Group II

ETOH+NAD+Na pyrophosphate+Na phosphate+alcohol dehydrogenase+aldehyde dehydrogenase

Time:

| | 0 | 30 min. | |
|---|---|---|---|
| Alcohol Concentration: | mg | mg | % reduction |
| Group I | 125 | 102 | 18.4 |
| Group II | 157 | 104 | 33.7 |

This example shows that by mixing alcohol with alcohol dehydrogenase or alcohol dehydrogenase combined with the aldehyde dehydrogenase one sees a reduction in alcohol concentration.

What is claimed is:

1. A method of moderating the buildup of alcohol in the blood of a patient following alcoholic beverage ingestion said method comprising administering to the patient of an effective amount of alcohol dehydrogenase sufficient to reduce the blood alcohol level in said patient.

2. The method of claim I wherein the alcohol dehydrogenase is combined with aldehyde dehydrogenase, said treatment to reduce blood alcohol as well as a toxic metabolite buildup, of aldehyde.

3. The method of claim I wherein the alcohol dehydrogenase and the aldehyde dehydrogenase is administered before or with ingestion of alcohol.

4. The method of claim I wherein an anti-cholinergic agent is administered along with the alcohol dehydrogenase to minimize gastric emptying to thereby allow more time for said agent to inactivate the alcohol before gastric emptying occurs.

5. The method of claim 1 and 2 wherein a treatment consisting of alcohol and aldehyde dehydrogenase within a wax or plastic matrix to reduce the effects of gastric pH on the integrity of the alcohol or aldehyde dehydrogenase enzyme treatment.

6. The method of claims I+2 wherein a treatment is administered of alcohol and aldehyde dehydrogenase administered along with an anti-cholinergic or the addition of an antacid to moderate gastric pH to optimize enzymatic protection and breakdown of the alcohol or aldehyde dehydrogenase.

7. The method of claim 1 & 2 wherein a formulation of alcohol dehydrogenase and aldehyde dehydrogenase administered with an antacid or buffer be it solid or liquid that would increase the pH of gastric secretions thus protecting the alcohol and aldehyde dehydrogenase.

8. The method in claim 1 & 2 wherein a formulation of alcohol dehydrogenase and/or aldehyde dehydrogenase administered with an H₂ blocker or a proton pump inhibitor alone or in combination with an antacid or H₂ blocker to diminish the acid content of the gastric secretions preventing degradation of the enzymes.

9. The method of claims 1 & 2 wherein formulation of alcohol dehydrogenase and/or aldehyde dehydrogenase administered with an H₂ blocker or proton pump inhibitor in combination with an anti-cholinergic drug to raise gastric pH and decrease gastric emptying, prolonging the time for the enzyme to metabolize the alcohol and aldehyde while diminishing the effect of gastric acid on the enzymes integrity.

10. The method of claims 1 and 2 wherein a formulation of alcohol dehydrogenase and/or aldehyde dehydrogenase is administered with an H₂ blocker or proton pump inhibitor in combination with an anti-cholinergic drug with the addition of NDA, Na pyrophosphate and Ne phosphate.
